(19) United States
(12) Patent Application Publication
(10) Pub. No.: US 2003/0220369 A1
(43) Pub. Date: Nov. 27, 2003

(54) Heterocyclic sulfonamide derivatives and their use for potentiating glutamate receptor function

(76) Inventors: Scott Louis Forman, Indianapolis, IN (US); Winton Dennis Jones, Carmel, IN (US); Daryl Lynn Smith, Fishers, IN (US); Hamideh Zarrinmayeh, Carmel, IN (US); Dennis MIchael Zimmerman, Zionsville, IN (US)

Correspondence Address: ELI LILLY AND COMPANY, PATENT DIVISION, P.O. BOX 6288, INDIANAPOLIS, IN, 46206-6288, US

(21) Appl. No.: 10/332,941

(22) PCT Filed: Jul. 27, 2001
(86) PCT No.: PCT/US01/21122

(51) Int. Cl. A61K 31/4706; A61K 31/428; A61K 31/423; A61K 31/4184; A61K 31/405; C07D 215/20; C07D 263/58; C07D 277/62

(52) U.S. Cl. 514/312; 514/367; 514/375; 514/394; 514/418; 546/158; 548/165; 548/221; 548/306.4; 548/484

(57) ABSTRACT

The present invention provides certain heterocyclic sulfonamide derivatives of formula I: useful for potentiating glutamate receptor function in a patient and therefore, useful for treating a wide variety of conditions, such as psychiatric and neurological disorders.

### BACKGROUND OF THE INVENTION

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathway in mammals, and is referred to as an excitatory amino acid (EAA). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic”. This type of receptor has been subdivided into at least three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol Sci., 11, 508 (1990); McDonald and Johnson, Brain Research Reviews, 15, 41 (1990).

AMPA receptors are assembled from four protein sub-units known as GluR1 to GluR4, while kainic acid receptors are assembled from the sub-units GluR5 to GluR7, and KA-1 and KA-2. Wong and Mayer, Molecular Pharmacology 44: 505-510, 1993. It is not yet known how these sub-units are combined in the natural state. However, the structures of certain human variants of each sub-unit have been elucidated, and cell lines expressing individual sub-unit variants have been cloned and incorporated into test systems designed to identify compounds which bind to or interact with them, and hence which may modulate their function. Thus, European patent application, publication number EP-A2-0574257 discloses the human sub-unit variants GluR1B, GluR2B, GluR3A and GluR3B. European patent application, publication number EP-A1-0583917 discloses the human sub-unit variant GluR4B.

One distinctive property of AMPA and kainic acid receptors is their rapid deactivation and desensitization to glutamate. Yamada and Tang, The Journal of Neuroscience, September 1993, 13(9): 3904-3915 and Kathryn M. Partin, J. Neuroscience, Nov. 1, 1996, 16(21): 6634-6647.

It is known that the rapid desensitization and deactivation of AMPA and/or kainic acid receptors to glutamate may be inhibited using certain compounds. This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

International Patent Application Publication WO 98/33496 published, Aug. 6, 1998 discloses certain sulfonamide derivatives which are useful, for example, for treating psychiatric and neurological disorders, for example cognitive disorders; neuro-degenerative disorders such as Alzheimer's disease; age-related dementias; age-induced memory impairment; movement disorders such as tardive dyskinesia, Huntington's chorea, myoclonus, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, and drug-induced psychosis.

### SUMMARY OF THE INVENTION

The present invention provides compounds of formula I:

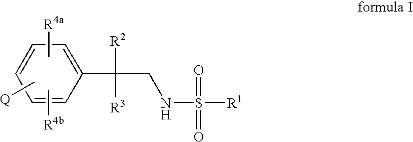

wherein

R¹ represents (1-6C)alkyl, (2-6C)alkenyl, or NR⁷R⁸;

R² and R³ each independently represent hydrogen, F, (1-4C)alkyl, or

—OR⁹;

R^{4a} and R^{4b} each independently represent hydrogen, (1-4C) alkyl, (1-4C)alkoxy, I,

Br, Cl, or F; and

Q is selected from the following:

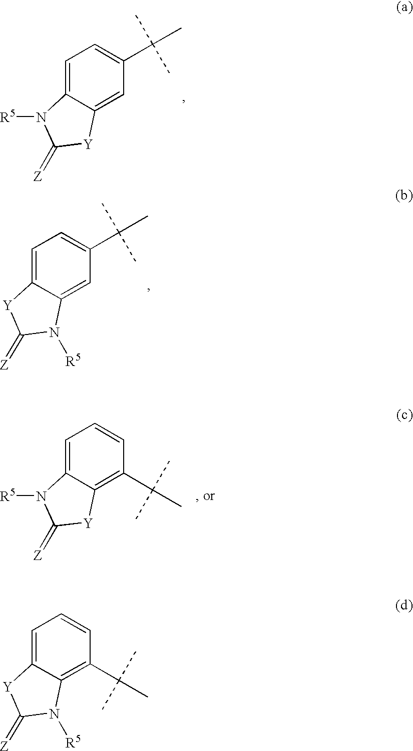

wherein

R⁵ represents hydrogen or (1-6C)alkyl;

Y represents CH₂CH₂, CR¹⁰R¹¹, NR⁶, S, or O;

Z represents O, S, or NH; and

R⁶ represents hydrogen or (1-6C)alkyl;

R⁷ and R⁸ each independently represent hydrogen or (1-4C)alkyl;

R⁹ represents hydrogen or (1-4C)alkyl; and

R¹⁰ and R¹¹ each independently represent hydrogen or (1-4C)alkyl;

or a pharmaceutically acceptable salt thereof;

with the proviso, that at least one of R² and R³ represents F, and with the further proviso that the compound of formula I is other than (±)-5-[4-(1-fluoro-1-methyl-2-{[(methylethyl)sulfonyl]amino}ethylphenyl]-indolin-2-one or (±)-6-[4-(1-fluoro-1-methyl-{[(methylethyl)sulfonyl]amino}ethylphenyl]-indolin-2-one.

The present invention further provides a method of potentiating glutamate receptor function in a patient, which comprises administering to said patient an effective amount of a compound of formula I.

The present invention provides a method of treating cognitive disorders in a patient, which comprises administering to said patient an effective amount of a compound of formula I.

In addition, the present invention further provides a method of treating cognitive deficits associated with psychosis in a patient, which comprises administering to said patient an effective amount of a compound of formula I.

According to another aspect, the present invention provides the use of a compound of formula I, or a pharmaceutically acceptable salt thereof for the manufacture of a medicament for potentiating glutamate receptor function.

In addition, the present invention provides the use of a compound of formula I or a pharmaceutically acceptable salt thereof for potentiating glutamate receptor function.

The invention further provides pharmaceutical compositions comprising, a compound of formula I and a pharmaceutically acceptable diluent or carrier.

This invention also encompasses novel intermediates, and processes for the synthesis of the compounds of formula I.

### DETAILED DESCRIPTION OF THE INVENTION

In this specification, the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

As used herein the term “AMPA receptor potentiator” refers to a compound which inhibits the rapid desensitization or deactivation of AMPA receptors to glutamate.

A wide variety of conditions may be treated or prevented by compounds of formula I and their pharmaceutically acceptable salts through their action as potentiators of glutamate receptor function. Such conditions include those associated with glutamate hypofunction, such as psychiatric and neurological disorders, for example cognitive disorders and neuro-degenerative disorders such as Alzheimer's disease; age-related dementias; age-induced memory impairment; cognitive deficits due to autism, Down's syndrome and other central nervous system disorders with childhood onset, cognitive deficits post electroconvulsive therapy, movement disorders such as tardive dyskinesia, Hungtington's chorea, myoclonus, dystonia, spasticity, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, drug-induced psychosis, stroke, and sexual dysfunction. Compounds of formula I may also be useful for improving memory (both short term and long term) and learning ability. The present invention provides the use of compounds of formula I for the treatment of each of these conditions.

The present invention includes the pharmaceutically acceptable salts of the compounds defined by formula I. A compound of this invention can possess a sufficiently acidic group, a sufficiently basic group, or both functional groups, and accordingly react with any of a number of organic and inorganic-bases, and inorganic and organic acids, to form a pharmaceutically acceptable salt. The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts. Such salts include the pharmaceutically acceptable salts listed in Journal of Pharmaceutical Science, 66, 2-19 (1977) which are known to the skilled artisan. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprate, caprylate, acrylate, ascorbate, formate, hydrochloride, dihydrochloride, isobutyrate, caproate, heptanoate, propiolate, propionate, phenylpropionate, salicylate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydroxybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, benzenesulfonate, methanesulfonate, ethanesulfonate, propanesulfonate, hydroxyethanesulfonate, 1-naphthalenesulfonate, 2-napththalenesulfonate, 1,5-naphthalenedisulfonate, mandelate, tartarate, and the like. Preferred pharmaceutically acceptable acid addition salts are those formed with mineral acids such as hydrochloric acid and hydrobromic acid, and those formed with organic acids such as maleic acid, oxalic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ⅇⅇ = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates, and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

As used herein the term “(±)-5-[4-(1-fluoro-1-methyl-2-{[(methylethyl)sulfonyl]amino}ethylphenyl]-indolin-2-one” refers to the racemic mixture of a compound with the following structure:

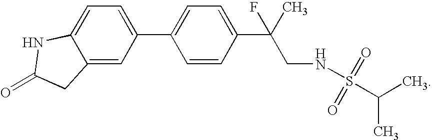

As used herein the term “(±)-6-[4-(1-fluoro-1-methyl-{[(methylethyl)sulfonyl]amino}ethylphenyl]-indolin-2-one” refers to the racemic mixture of a compound with the following structure:

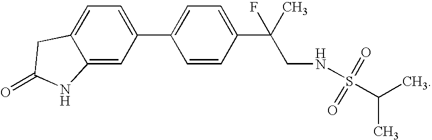

As used herein the term “(1-6C)alkyl” refers to straight or branched, monovalent, saturated aliphatic chains of 1 to 6 carbon atoms and includes, but is not limited to, methyl, ethyl, propyl, isopropyl, butyl, isobutyl, t-butyl, pentyl, isopentyl, and hexyl. The term “(1-6C)alkyl” includes within its definition the term “(1-4C)alkyl”.

As used herein the term “(2-6C)alkenyl” refers to a straight or branched, monovalent, unsaturated aliphatic chain having from two to six carbon atoms and includes, but is not limited to, ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein the term “(1-4C)alkoxy” refers to a straight or branched alkyl chain having from one to six carbon atoms attached to an oxygen atom, and includes methoxy, ethoxy, propoxy, isopropoxy, butoxy, t-butoxy, and the like.

The terms “halogen”, “Hal” or “halide” include fluorine, chlorine, bromine and iodine unless otherwise specified.

As used herein the term “bis(pinacolato)diboron” refers to the following structure:

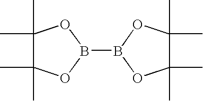

The compounds of formula I can be prepared by one of ordinary skill in the art following, for example, the various procedures set forth in the Schemes below. The reagents and starting materials are readily available to one of ordinary skill in the art, for example, see International Patent Application Publications: WO 98/33496 published Aug. 6, 1998, and WO 00/06148 and WO 00/06158, both published Feb. 10, 2000. All substituents, unless otherwise specified are as previously defined.

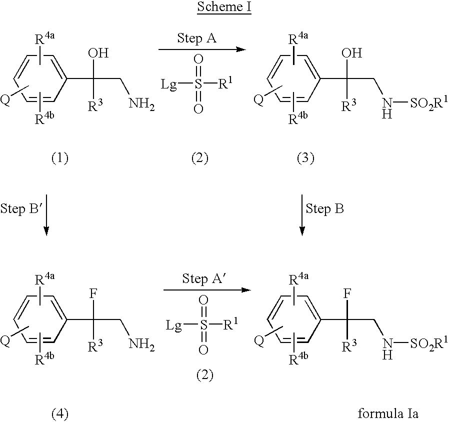

In Scheme I, step A the compound of structure (1) is combined with the compound of structure (2) under conditions well known in the art to provide the compound of structure (3). More specifically, for example, the compound (1) is dissolved in a suitable organic solvent. Examples of suitable organic solvents include methylene chloride, tetrahydrofuran, and the like. The solution is treated with a slight excess of a suitable base, and then cooled to about −78° C. to about 0° C. Examples of suitable bases include triethylamine, pyridine, 1,8-diazabicyclo[5.4.0]undec-7-ene (DBU), and the like. To the stirring solution is added one equivalent of compound (2). The term “Lg” as used herein refers to a suitable leaving group. Examples of suitable leaving groups include, Cl, Br, and the like. Cl is the preferred leaving group. The reaction mixture is stirred at about 0° C. to about 50° C. for about 0.5 hours to about 16 hours. The compound (3) is then isolated and purified by techniques well known in the art, such as extraction techniques and chromatography. For example, the mixture is washed with 10% sodium bisulfate, the layers separated and the aqueous extracted with several times with a suitable organic solvent, such as methylene chloride. The organic extracts are combined, dried over anhydrous sodium sulfate, filtered and concentrated under vacuum. The residue is then purified by flash chromatography on silica gel with a suitable eluent such as ethyl acetate/hexane to provide the compound (3).

In Scheme I, step B the compound of structure (3) is fluorinated under conditions well known in the art to provide the compound of formula Ia. For example, compound (3) is dissolved in a suitable organic solvent, such as methylene chloride and the solution is cooled to about −78° C. under an inert atmosphere, such as nitrogen. To this solution is added slowly, about one equivalent of diethylaminosulfur trifluoride (DAST) dissolved in a suitable organic solvent, such as methylene chloride with stirring. The reaction is then allowed to warm to room temperature (about 22° C.) and the compound of formula Ia is then isolated and purified using techniques and procedures well known in the art, such as extraction techniques and chromatography. For example, the reaction is diluted with water and methylene chloride. The layers are separated and the organic layer is washed with water, dried over anhydrous sodium sulfate, filtered and concentrated under vacuum to provide the crude compound of formula Ia. This crude material can then be purified by standard techniques, such as recrystallization from a suitable eluent, or chromatography on silica gel, with a suitable eluent, such as hexane/ethyl acetate to provide purified compound of formula Ia.

Alternatively, in Scheme I, step B′ the compound (1) is fluorinated in a manner analogous to the procedure described in step B above with DAST to provide the compound of structure (4). In Scheme I, step A′ compound (4) is converted to the compound of formula Ia in a manner analogous to the procedure described in step A above.

The compounds of formula Ia can be prepared following the procedure described in Scheme II. The reagents and starting materials are readily available to one of ordinary skill in the art. All substituents, unless otherwise specified are as previously defined.

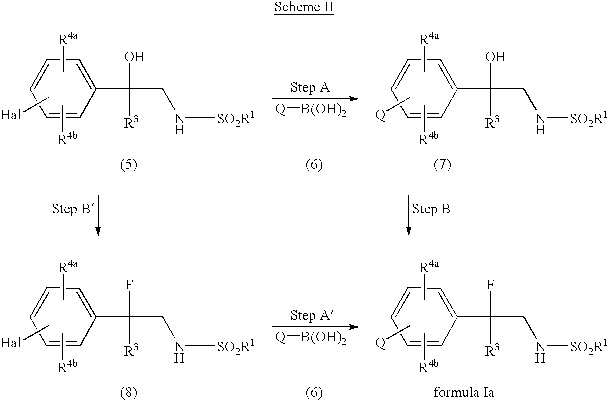

In Scheme II, step A the compound of structure (5) is coupled with compound of structure (6) under standard Suzuki coupling conditions to provide compound of structure (7). See Suzuki, A., Journal of Organometallic Chemistry, 576, 147-168 (1999), and Miyaura and Suzuki, Chemical Reviews, 95, 2457-2483 (1995) for examples of Suzuki-type coupling reactions and conditions. For example, compound (5) is combined with about 1.5 equivalents of compound (6), about 1.5 equivalents of potassium carbonate, and about 0.06 equivalents of tetrakis(triphenyl phosphine)palladium(0) in a suitable solvent or solvent mixture, such as dioxane/water (3:1). The mixture is then heated at about 100° C. for about 18 hours. The reaction is then cooled and compound (7) is isolated and purified using standard techniques and procedures, such as extraction techniques and chromatography. For example, the reaction mixture is extracted with a suitable organic solvent, such as ethyl acetate, the organic extracts are combined, washed with water, dried over anhydrous sodium sulfate, filtered, and concentrated under vacuum. The crude material is then purified by chromatography on silica gel with a suitable eluent, such as hexane/ethyl acetate to provide purified compound (15).

In Scheme II, step B, compound (7) is fluorinated under standard conditions to provide the compound of formula Ia. For example, compound (7) is dissolved in a suitable organic solvent, such as methylene chloride is added to about one equivalent of DAST at about −78° C. with stirring under an atmosphere of nitrogen. The reaction is allowed to warm to room temperature and the compound of formula Ia is isolated and purified using standard techniques, such as extraction techniques and chromatography. For example, the reaction mixture is diluted with water and a suitable organic solvent, such as methylene chloride. The layers are separated and the organic layer is washed with water, dried over anhydrous sodium sulfate, filtered and concentrated under vacuum. The crude material is then purified by chromatography on silica gel with a suitable eluent, such as hexane/ethyl acetate to provide the purified compound of formula Ia.

Alternatively, in Scheme II, step B′ the compound (5) is fluorinated to provide the compound (8) in a manner analogous to the procedure described above in step B.

In addition, in Scheme II, step A′ the compound (8) is converted to the compound of formula Ia in a manner analogous to the procedure described above in step A.

Compounds of formula Ib can be prepared as shown in Scheme III. Reagents and starting materials are readily available to one of ordinary skill in the art. All substituents, unless otherwise specified, are previously defined.

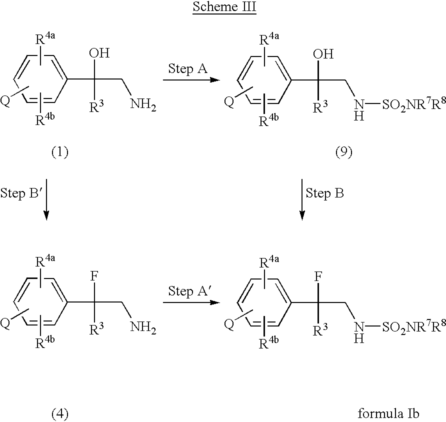

In Scheme III, step A, the compound of structure (1) is combined with a compound of formula CISO₂NR⁷R⁸ under standard conditions to provide the compound of structure (9). For example, compound (1) is dissolved in a suitable organic solvent, such as tetrahydrofuran and treated with about one equivalent of a suitable base, such as DBU at about 0° C. The solution is then treated with about one equivalent of a compound of formula CISO₂NR⁷R⁸. The reaction is then allowed to warm to room temperature and stirred for about 4 to 16 hours. The reaction is then concentrated under vacuum to provide the crude product (9) which can then be purified by chromatography on silica gel with a suitable eluent, such as ethyl acetate/hexane.

In Scheme III, step B, compound (9) is converted to the compound of formula Ib in a manner analogous to the procedure set forth in Scheme I, step B.

Alternatively, in Scheme III, step B′ the compound (1) is fluorinated in a manner analogous to the procedure described in Scheme I, step B with DAST to provide the compound of structure (4). In Scheme III, step A′ compound (4) is converted to the compound of formula Ib in a manner analogous to the procedure described above in step A.

The compounds of structure (5) can be prepared following the procedure described in Scheme IV. The reagents and starting materials are readily available to one of ordinary skill in the art. All substituents, unless otherwise specified are as previously defined.

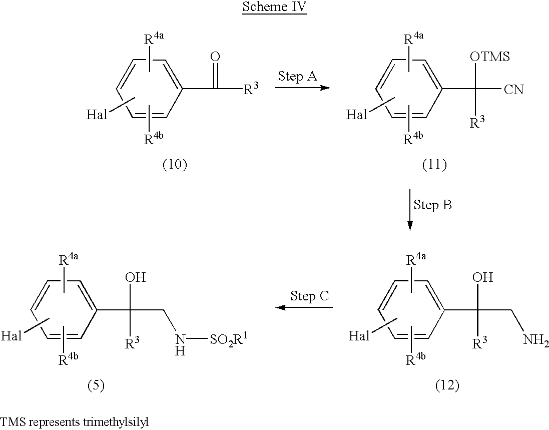

In Scheme IV, step A the compound of structure (10) is converted to the compound of structure (11) under standard conditions. For example, see Greenlee and Hangauer, Tetrahedron Lett., 24(42), 4559 (1983). For example, compound (10) is dissolved in a suitable organic solvent, such as dry tetrahydrofuran, containing excess 18-crown-6, and excess potassium cyanide. To this mixture at room temperature is added dropwise about 1.2 equivalents of cyanotrimethylsilane. The reaction mixture is allowed to stir for about 1 to 4 hours to provide compound (11). Compound (11) is then carried on directly to step B without isolation.

Alternatively, in Scheme IV, step A, for example, compound (10) is combined with a catalytic amount of zinc iodide followed by slow addition of excess trimethylsilyl cyanide with the generation of heat. The resulting solution is stirred at room temperature under nitrogen for about 8 to 16 hours. The mixture is then diluted with a suitable organic solvent, such as chloroform, washed with saturated sodium bicarbonate, water, brine, dried over anhydrous magnesium sulfate, filtered, and concentrated under vacuum to provide compound (11).

In Scheme IV, step B compound (11) is converted to compound of structure (12). For example, compound (11) prepared above, still in solution, is treated with a solution of about 1.4 equivalents of borane in dimethylsulfide. The reaction mixture is then heated to reflux for about 16 hours and then cooled to room temperature. The reaction mixture is then cautiously treated with anhydrous HCl in methanol and allowed to stir for about one hour. The product (12) is then isolated and purified using standard techniques and procedures. For example, the solvent is removed under vacuum and the residue triturated with a suitable organic solvent, such at methy t-butyl ether and the solid is collected by filtration. The solid is then suspended in methylene chloride/tetrahydrofuran mixture (1:2.4) and treated with IN NaOH until about pH 12.3 is reached. The phases are separated and the organic phase is rinsed with brine. The organic phase is then concentrated under vacuum and the residue triturated with diethyl ether to provide the purified compound (12).

In Scheme IV, step C, compound (12) is then sulfonylated to provide compound (5) in a manner analogous to the procedure described in Scheme I, step A.

The following examples further illustrate the invention and represent typical syntheses of the compounds of formula I as described generally above. The reagents and starting materials are readily available to one of ordinary skill in the art. The reagents and starting materials are readily available to one of ordinary skill in the art. As used herein the term “Chromatotron®” (Harrison Research Inc., 840 Moana Court, Palo Alto Calif. 94306) is recognized by one of ordinary skill in the art as an instrument which is used to perform centrifugal thin-layer chromatography. As used herein, the following terms have the meanings indicated: “eq” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “EtOAc” refers to ethyl acetate; “KOAc” refers to potassium acetate; “aq” refers to aqueous; “iPrOAc” refers to isopropyl acetate; “MTBE” refers to tert-butyl methyl ether; “methyl DAST” refers to dimethylaminosulfur trifluoride, “DAST” refers to diethylaminosulfur trifluoride, “DBU” refers to 1,8-diazabicyclo[5.4.0]undec-7-ene; as used herein “Pd(dppf)₂Cl₂ catalyst” refers to ([1,1′-bis(diphenylphosphino)ferrocene]dichloropalladium(II) complex with CH₂Cl₂; as used herein the term “NBS” refers to N-bromosuccinimde; as used herein the terms “Me”, “Et”, “Pr”, “iPr”, and “Bu” refer to methyl, ethyl, propyl, isopropyl, and butyl respectively, and “RT” refers to room temperature.

### Preparation 1

Preparation of 6-Bromo-3,4-dihydro-1H-quinolin-2-one.

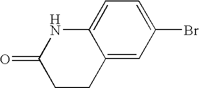

To a stirred solution of 3,4-dihydro-1H-quinolin-2-one (0.735 g, 5.0 mmol) in dry DMF (20.0 mL) under N₂ at ambient temperature was added N-bromosuccimide (0.93 g, 5.2 mmol) portionwise. The solution was stirred under nitrogen overnight and then the orange mixture was poured into H₂O (200 mL). The precipitated solid was extracted into ether(100 mL). The ether was separated, extracted with H₂O (4×150 mL), washed with brine and dried(MgSO₄). Filtration and evaporation in vacuo gave the title compound (0.70 g, 62%) as a light tan powder.

¹H NMR(CDCl₃) δ2.60 (2H, m), 2.90 (2H, m), 6.65 (1H, d), 7.2-7.3 (2H, m); 8.9(1H, br s).

### Preparation 2

Preparation of 6-(4,4,5,5-Tetramethyl-[1,3,2]dioxaborolan-2-yl)3,4-dihydro-1H-quinolin-2-one.

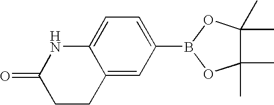

6-Bromo-3,4-dihydro-1H-quinolin-2-one (0.35 g, 1.35 mmol) was combined with pinacol borane (0.30 g, 2.33 mmol), [1,1′-bis(diphenylphosphino)ferrocene]dichloropalladium(II) complex with dichloromethane (0.04 g, 0.05 mmol) and triethylamine (0.65 mL, 4.65 mmol) in dry acetonitrile (7 mL) and heated at reflux under N₂ for 4 hours then cooled to ambient temperature. The reaction mixture was dumped into diethyl ether and washed with water and saturated NaCl, dried (MgSO₄), filtered, and concentrated to give the title compound (0.44 g) as a light yellow oil which was used without purification.

### Preparation 3

Preparation of 6-Bromo-3H-benzoxazol-2-one.

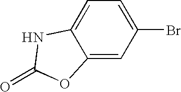

To a stirred suspension of 3H-benzoxazol-2-one (1.35 g, 10 mmol) in acetonitrile (23 mL ) at −15° C. was added portionwise NBS (2.00 g, 11.0 mmol). Following complete addition of NBS the mixture was stirred at −15 to 0° C. for 3 h then allowed to warm to ambient temperature and stirred overnight. The solvent was evaporated in vacuo and the residue was partitioned between CH₂Cl₂/H₂O precipitating the title compound, 6-bromo-3H-benzoxazol-2-one, (0.67 g, 31%) as a brown solid. ¹HNMR(CDCl₃) δ7.0 (1H, d), 7.15 (1H, d); 7.3 (1H, s), 11.9 (1H, s).

### Preparation 4

Preparation of 5-Bromo-1,3-dihydro-indol-2-one.

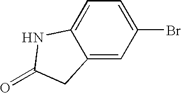

5-Bromo-1,3-dihydro-indol-2-one can be prepared following the procedure described by Sun et al., J. Med. Chem., 41, 2588-2603 (1998). For example, to a stirred suspension of oxindole (1.30 g, 10 mmol) in dry acetonitrile(22.0 mL) at −10° C. was added portionwise recrystallized NBS (2.00 g, 11.0 mmol) and the resulting suspension was stirred at −10 to 0° C. for 3 hours. The suspension was allowed to warm to ambient temperature and the mixture was filtered to give a white solid which was recrystallized (EtOH) to provide the title compound (1.47, 43%) as a slightly pink solid; mp 212-2140° C.

### Preparation 5

Preparation of 7-Bromo-1,3-dihydro-indol-2-one.

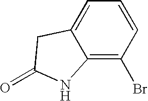

To a freshly prepared mixture of 2-nitro-6-bromo-phenyl acetic(1.98 g, 7.61 mmol) in EtOH (20 mL) and 9M H₂SO₄ (20 mL) at 50° C. was added Zn dust(2.00 g, 30.6 mmol) at ambient temperature. The reaction was stirred for 30 minutes at ambient temperature and then at 70° C. for 30 minutes. The reaction was cooled to ambient temperature and poured into H₂O (100 mL) and estracted with EtOAc (2×50 mL). The combined organic layers were dried (Na₂SO4), filtered and evaporated to give a crude brown residue. Chromatography on silica gel eluting with a gradient EtOAc-hexane system (1:10 to 1:1) gave the title compound (0.43 g, 27%) as a light brown solid.

Analysis

| | |
|---|---|
| Theory: | C, 45.31; H, 2.85; N, 6.60 |
| Found: | C, 45.01; H, 2.55; N, 6.42 |

### Preparation 6

Preparation of 6-Bromo-1,3-dihydro-indol-2-one.

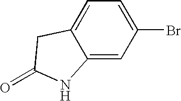

To a stirred solution of 4-bromo-2-nitrophenylacetic acid (1.00 g, 3.85 mmol) dissolved in 50% H₂SO₄ (8.2 mL)/EtOH (10.9 mL) was added Zn dust (1.01 g, 15.38 mmol) at 90° C. under nitrogen. The reaction was then treated in a manner analogous to the cyclization described above in preparation 5 to provide the title compound (0.512 g, 63%); mp 167-169° C.

### Preparation 7

Preparation of 6-Bromo-3-methyl indol-2-one.

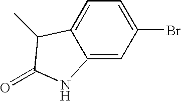

To a stirred solution of 2-(4-bromo-2-nitro-benzene)propionic acid(0.800 g, 2.91 mmol) dissolved in 50% H₂SO4 (6.2 mL)/EtOH (8.5) was added Zn dust (0.76 g, 11.6 mmol) at 90° C. under N₂. The reaction mixture was then treated in a manner analogous to preparation 5 to provide the title compound (0.61 g, 93%).

Analysis

| | |
|---|---|
| Theory: | C, 47.83; H, 3.57; N, 6.20 |
| Found: | C, 47.80; H, 3.78; N, 5.83 |

### Preparation 8

Preparation of 2-(4-Bromo-2-nitro-phenyl)propionic acid.

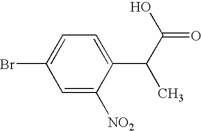

A mixture of 3-bromo-nitrotoluene (3.02 g, 12 mmol) and 2-chloro ethyl propionate (1.91 mL, 15.O mmol) in anhydrous THF (15.0 mL) was added dropwise to a solution of 1.0 M KOtBu (30.0 mL) under N₂ at −40° C. over 10 min. The reaction turned a deep blue color. It was stirred at −45 to −35° C. for 1 h then allowed to warm to −10° C. It was then cooled again to −40° C. and quenched with 2 M HCl (20 mL). The mixture was diluted with EtOAc(175 mL). The organic layer was washed with brine, separated and dried (MgSO₄). Filtration followed by evaporation of the filtrate in vacuo gave a brown oil. The oil was placed onto silica gel in a 300 mL sintered glass funnel and eluted with EtOAc (1 L). Evaporation of the filtrate gave 1.45 g (40%) of the intermediate ester as a light brown oil. TLC(EtOAc-hexane 1:4), R_{f}=0.32. To a stirred solution of the ester in EtOH (10 mL) under N₂. was added a 5.26% solution of NaOH (28 mL) at ambient temperature. The resulting dark brown reaction mixture was stirred overnight at ambient temperature then poured into H₂O (250 mL). The aqueous mixture was extracted with Et₂O (2×100 mL). The aqueous layer was separated and acidified to PH 1. The oily precipitate was extracted into EtOAc. The organic layer was separated washed with brine separated, filtered and dried (MgSO₄). Filtration and evaporation gave the title compound (0.83 g) as a waxy solid. MS(ES): 275.1(M+1).

### Preparation 9

Preparation of 2-bromo-6-nitro-phenyl acetic acid.

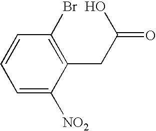

2-Bromo-6-nitro-phenyl acetic acid can be prepared according to the procedure of Magnus et al Tetrahedron Letters, 2,000, 835-838. For example, pyrrolidine (1.10 mL, 0.013 mol) was added to a stirred solution of 2-bromo-6-nitrotoluene (3.24 g, 0.015 mol) and dimethylformamide dimethyl acetal in dry DMF (20 mL) at ambient temperature under N₂. The resulting mixture heated and stirred for 3h at 110° C. The dark brown reaction mixture was poured into Et₂O—H₂O. The organic layer was extracted with H₂O, washed with brine, dried (MgSO₄), filtered and the filtrate evaporated. The brown residue was dissolved in a solution of THF (1O0 mL) at O° C. To this solution was added 1.6 M HCl (50 mL) and the resulting mixture was stirred at 0-10° C. for 1 h then allowed to warm to ambient temperature and stirred for an hour and a half at ambient temperature. The mixture was poured into H₂O (400 mL) and extracted with Et₂O. The organic layer was dried (MgSO₄), filtered and evaporated to give a brown oil. The oil was dissolved in acetone (100 mL). To this solution at 0-10° C. was added a 10% solution of NaClO₂ (75 mL) and sulfamic acid(10.72 g, 0.104 mol) in H₂O (100 mL) followed by 10% Na₂HPO4 (75 mL). The resulting mixture was stirred at 0-10° C. for 1 h and poured into H₂O (300 mL). The aqueous mixture was extracted with Et₂O (200 mL). The organic layer was separated and extracted NaHCO₃ (2×100 mL). The aqueous layer was acidified conc. HCl and extracted with Et₂O (200 mL). The Et₂O layer was washed with brine, dried (MgSO₄), filtered and evaporated to give the title acid (0.78 g, 20%) as a pale yellow solid mp 159-161° C.(dec.).

### Preparation 10

Preparation of 4-Bromo-1,3-dihydro-indol-2-one.

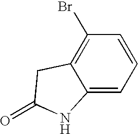

To a stirred solution of 2-bromo-6-nitro-phenyl acetic acid (0.66 g, 2.54 mmol) dissolved in 50% H₂SO4 (6.0 mL)/EtOH (10.0 mL) was added Zn dust (0.66 g, 10.1 mmol) at 90° C. under N₂. The reaction mixture was then treated in a manner analogous to the procedure described in preparation 5 to provide the title compound (0. 50 g, 93 MS(ES) M+1 212, M+2 214

### EXAMPLE 1

Preparation of [2-fluoro-2-(4-iodophenyl)propyl][(methylethyl)sulfonyl]amine.

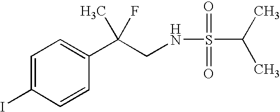

Preparation of 1-amino-2-(4-iodophenyl)propan-2-ol.

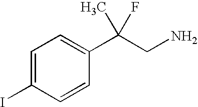

Scheme IV, step A: The trimethylsilyl-protected cyanohydrin derivative of 4-iodoacetophenone was prepared in situ following generally the method disclosed by Greenlee and Hangauer, Tetrahedron Lett., 24(42), 4559 (1983). Accordingly, cyanotrimethylsilane (21.4 g, 0.216 mol) was added dropwise over 5 minutes to a dry, room temperature solution containing 4-iodoacetophenone (44.3 g, 0.180 mol), 18-resulting semi-solid was purified via silica crown-6 (1.6 g, 6.1 mmoles) and KCN (1.17 g, 0.018 mol) in THF (100 mL). The resulting solution was allowed to stir for 2.5 h. TLC analysis (3:7 EtOAc/Hexanes) showed consumption of starting acetophenone.

Scheme IV, step B: A 10M solution of borane in dimethylsulfide (25 mL, 0.25 mol) was added rapidly to the reaction solution and the resulting mixture was heated at reflux for 16 h. The mixture was cooled to room temperature and anhydrous 10% (by wt) HCl in methanol was added slowly over 1 h (GAS EVOLUTION). The solution was allowed to stir for an additional hour, and was concentrated under reduced pressure to give the crude title compound as white solid and as the hydrochloride salt. This salt was triturated with methyl t-butyl ether and filtered. The free base was prepared by adding 1N NaOH to a suspension of the HCl salt in CH₂Cl₂ (150 mL) and THF (350 mL) until pH 12.3 was reached. The phases were separated and the organic phase was washed with brine (25 mL). The organic phase containing the free amine was concentrated under reduced pressure and the resulting solids were triturated with diethyl ether (30 mL) to afford the intermediate title compound (35.6 g, 71.3%) as an off-white powder after vacuum drying. ¹H NMR (CD₃OD, 300 MHz): δ7.68 (d, 2H, J=8.4), 7.24 (d, 2H, J=8.7), 2.78 (m, 2H), 1.46 (s, 3H).

Preparation of [2-hydroxy-2-(4-iodophenyl)propyl][(methylethyl)sulfonyl]amine.

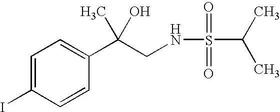

Scheme IV, step C: Into a 250 mL 3 necked flask fitted with a stirrer and thermometer, was added dropwise 2-propanesulfonyl chloride (1.60 g, 0.011 mol) to 1-amino-2-(4-iodophenyl)propan-2-ol (2.77 gm, 0.01 mol) in 125 mL CH₂Cl₂ while stirring at 0° C. under nitrogen. The reaction was then allowed to warm to room temperature and stirred overnight at this temperature. In the morning, the mixture was poured into H₂O and the layers were separated. The organic layer was washed once with H₂O, dried over anhydrous Na₂SO₄, filtered, and concentrated under reduced vacuum. The gel chromatography employing the Prep. LC-2000 and eluting with a solvent of Hexane/EtOAc 3:1 to provide the intermediate title compound (744 mg, 19%) as a solid material. FDMS 382 (M*).

Analysis for C₁₂H₁₈NO₃ S I:

| | |
|---|---|
| Theory: | C, 37.61 H, 4.73 N, 3.65 |
| Found: | C, 38.08 H, 4.26 N, 3.55 |

Alternative preparation of [2-hydroxy-2-(4-iodophenyl)propyl][(methylethyl)sulfonyl]amine.

Scheme IV, step C: In a 250 mL-3 neck flask fitted with a stirrer and thermometer, 2.10 g. of propanesulfonyl chloride was added dropwise to 2.77 g. of 1-amino-2-(4-iodophenyl)propan-2-ol and 2.30 g. of DBU in CH₂Cl₂ (150 mL) while stirring at 0° C. under a nitrogen atmosphere. The reaction was allowed to warm to room temperature and stirred overnight at this temperature. In the morning, the reaction was diluted with CH₂Cl₂ (100 mL) and the organic layer was washed two times with H₂O, dried over anhydrous Na₂SO₄, filtered, and concentrated under reduced vacuum to yield a viscous oil. This material was purified via silica gel chromatography employing the Chromatotron®, using a 4000 micron rotor and eluting with a solvent of methylene chloride/methanol 19:1 to yield the intermediate title compound (1.0 g, 31%) as a viscous oil. Ion spray M.S. 382 (M*−1).

Preparation of final title compound.

Scheme I, step B: Into a 10 mL single neck flask, a solution of [2-hydroxy-2-(4-iodophenyl)propyl][(methylethyl)sulfonyl]amine (158 mg, 0.41 mmol) in 1.7 mL CH₂Cl₂ was added syringe wise slowly to a solution of DAST (66 mg, 0.41 mmol) in 0.3 mL CH₂Cl₂ while stirring at −78° C. under nitrogen. The reaction was then allowed to warm to room temperature and the mixture was diluted with H₂O and CH₂Cl₂. The layers were separated and the organic layer was washed twice with H₂O, dried over anhydrous Na₂SO₄, filtered, and concentrated under reduced vacuum to provide the final title compound (0.113 g) as a solid. Ion spray M.S. 384 (M*−1).

Additional preparation of final title compound.

Scheme I, step B: Into a 100 mL 3-neck flask fitted with a stirrer and thermometer, 1.0 g. of [2-hydroxy-2-(4-iodophenyl)propyl][(methylethyl)sulfonyl]amine in CH₂Cl₂ (15 mL) was added dropwise to 0.3 mL DAST in CH₂CL₂ (10 mL) while stirring at −78° C. under a nitrogen atmosphere. Reaction was allowed to warm to room temperature and diluted with CH₂Cl₂ (50 mL). This organic layer was washed with H₂O, dried over anhydrous Na₂SO₄, filtered, and concentrated under reduced vacuum to yield an oil. This material was purified via silica gel chromatography employing the Chromatotron®) and using a 4000 micron rotor while eluting with a gradient solvent of hexane/ethyl acetate 9:1 to hexane/ethyl acetate 3:1 to yield the final title compound (0.906 g) as a white solid. Ion spray M.S. 384 (M*−1).

Analysis for C₁₂H₁₇NO₂SFI:

| | |
|---|---|
| Theory: | C, 37.42 H, 4.44 N, 3.64 |
| Found: | C, 37.27 H, 4.33 N, 3.61 |

### EXAMPLE 1a

Preparation of (+)-[2-fluoro-2-(4-iodophenyl)propyl][(methylethyl)sulfonyl]amine and (−)-[2-fluoro-2-(4-iodophenyl)propyl][(methylethyl)sulfonyl]amine.

[2-fluoro-2-(4-iodophenyl)propyl][(methylethyl)sulfonyl]amine (2.0 g, prepared in example 1) was dissolved into 3A ethanol (30 mL) and was further diluted with heptane (20 mL).[As used herein the term “3A ethanol” refers to ethanol containing 5% methanol.] The mixture was agitated via ultrasound to form a clear, colorless solution. This lot was loaded upon a 8×28 cm preparative Chiralpak AD chromatographic column that was pre-equilibrated with 60% 3A ethanol/40% heptane. Eluent flow was 300 mL/min and detection wavelength was 240 nm. The first eluting substance was (+)-[2-fluoro-2-(4-iodophenyl)propyl][(methylethyl)sulfonyl]amine, [α]_{D}=+18.5 (c=1.08, MeOH), and the subsequent eluting substance was (−)-[2-fluoro-2-(4-iodophenyl)propyl][(methylethyl)sulfonyl]amine, [α]_{D}=−23.5 (c=1.02, MeOH). The above procedure was repeated twice in an analogous manner with [2-fluoro-2-(4-iodophenyl)propyl][(methylethyl)sulfonyl]amine (second run, 3.0 g dissolved in 50 mL 3A ethanol/heptane, 3:2 and a third run, 2.0 g dissolved in 0.8 g dissolved in 40 mL 3A ethanol/heptane, 3:2). Thus, in three runs, a total of 5.8 g of [2-fluoro-2-(4-iodophenyl)propyl][(methylethyl)sulfonyl]amine was resolved into its component enantiomers in the following yields after concentration (in vacuo) of fractions:

(+)-[2-fluoro-2-(4-iodophenyl)propyl][(methylethyl)sulfonyl]amine (2.38 g, 41.0%);

(−)-[2-fluoro-2-(4-iodophenyl)propyl][(methylethyl)sulfonyl]amine (1.2 g, 20.7%).

### EXAMPLE 2

Preparation of 5-[4-(1-fluoro-1-methyl-2-{[(methylethyl)sulfonyl]amino}ethylphenyl]-indolin-2-one (enantiomer 1).

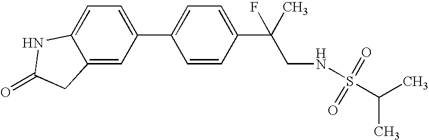

Scheme II, step A′: (+)-[2-fluoro-2-(4-iodophenyl)propyl][(methylethyl)sulfonyl]amine.(0.219 g, 0.57 mmol) bis(pinacolato)diboron (0.165 g, 0.65 mmol), PdCl₂(dppf).CH₂Cl₂ (0.024 g, 0.029 mmol) and potassium acetate (0.200 g, 2.0 mmol) were heated and stirred at 80° C. in dry DMF (14.0 mL) for 3 hours under N₂. The reaction mixture was allowed to cool to ambient temperature and 5-bromo-1,3-dihydro-indol-2-one (0.138 g, 0.651 mmol), PdCl₂(dppf).CH₂Cl₂ (0.025 g, 0.031 mmol), and 2M Na₂CO₃ (1.5, 3.0 mmol) were added respectively. Additional DMF (3.0 mL) was added . The resulting mixture was stirred and heated at 80° C. for 7 hours. The reaction mixture was allowed to cool to ambient temperature and poured into EtOAc(100 mL) and extracted with H₂O. The aqueous layer was separated and extracted with EtOAc. The organic layers were combined, washed with brine and dried (MgSO₄). Filtration and concentration in vacuo gave 0.226 g of a brown oil which when chromatographed on the Chromatotron® using a 4 mm plate and eluting with a gradient EtOAc/hexane 4:6 to 6:4 system provided the final title compound, (0.046 g, 21%) as a light tan crystalline solid, mp 149-151° C.

Analysis

| | |
|---|---|
| Theory: | C, 61.52; H, 5.94; N, 7.17 |
| Found: | C, 61.20; H, 5.60; N, 6.92 |

### EXAMPLE 3

Preparation of 5-[4-(1-fluoro-1-methyl-2-{[(methylethyl)sulfonyl]amino}ethylphenyl]-indolin-2-one (enantiomer 2).

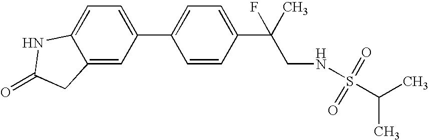

Scheme II, step A′: (−)-[2-fluoro-2-(4-iodophenyl)propyl][(methylethyl)sulfonyl]amine(0.438 g, 1.14 mmol) bis(pinacolato)diboron (0..330 g, 1.30 mmol), PdCl₂(dppf).CH₂Cl₂ (0.045 g, 0.055 mmol) and potassium acetate (0.400 g, 4.08 mmol) were heated and stirred at 80° C. in dry DMF (35.0 mL) for 3 hours under N₂. The reaction mixture was allowed to cool to ambient temperature and 5-bromo-1,3-dihydro-indol-2-one (0.138 g, 0.651 mmol), PdCl₂(dppf).CH₂Cl₂ (0.025 g, 0.031 mmol), and 2M Na₂CO₃ (1.5, 3.0 mmol) were added respectively. Additional DMF (3.0 mL) was added. The resulting mixture was stirred and heated at 80° C. for 7 hours. The reaction mixture was allowed to cool to ambient temperature and poured into EtOAc(100 mL) and extracted with H₂O. The aqueous layer was separated and extracted with EtOAc. The organic layers were combined, washed with brine and dried (MgSO₄). Filtration and concentration in vacuo gave 0.375 g of crude product which when chromatographed on the Chromatotron® using a 4 mm plate and eluting with a gradient EtOAc/hexane 4:6 to 6:4 system provided the final title compound (0.110 g, 21%) as an off white crystalline solid.

Analysis

| | |
|---|---|
| Theory: | C, 61.52; H, 5.94; N, 7.17 |
| Found: | C, 61.31; H, 5.83; N, 7.03 |

It is understood by one of ordinary skill in the art that the racemate of examples 2 and 3, 5-[4-(1-fluoro-1-methyl-2-{[(methylethyl)sulfonyl]amino}ethylphenyl]-indolin-2-one, is readily prepared in a manner analogous to the procedures described therein from racemic starting material.

### EXAMPLE 4

Preparation of 6-[4-(1-fluoro-1-methyl-{[(methylethyl)sulfonyl]amino}ethylphenyl]-indolin-2-one (enantiomer 1).

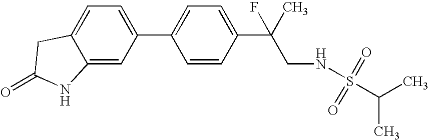

Scheme II, step A′: (+)-[2-fluoro-2-(4-iodophenyl)propyl][(methylethyl)sulfonyl]amine.(10.2, 26.47 mmol) bis(pinacolato)diboron (0.7.40 g, 29.14 mmol), PdCl₂(dppf).CH₂Cl₂(0.86 g, 1.05 mmol) and potassium acetate (9.04 g, 92.13 mmol) were heated and stirred at 80° C. in dry DMF (150.0 mL) for 6 hours under N₂. The reaction mixture was allowed to cool to ambient temperature and 6-bromo-1,3-dihydro-indol-2-one (6.18 g, 29.14 mmol)(preparation 6), PdCl₂(dppf).CH₂Cl₂ (0.86 g, 1.0 mmol.), and 2M Na₂CO₃ (40.0 mL, 80 mmol) were added respectively. The resulting mixture was stirred and heated at 80° C. for 20 hours. The reaction mixture was allowed to cool to ambient temperature and poured into EtOAc and extracted with H₂O. The aqueous layer was separated and extracted with EtOAc and Et₂O. The organic layers were combined and filtered through Celite®. The purple Celite® cake was washed repeatedly with EtOAc and Et₂O. The combined organic layers were washed with brine and dried (MgSO₄). Filtration and concentration in vacuo gave 40.0 g of a dark brown oil. Flash chromatography using a gradient system consisting of 1Lhexane, 2L EtOAclhexane 3:7, 2L EtOAc/hexane1:1, 4L EtOAc/hexane7:3 after evaporation gave 9.1 g of a-yellow brown oil. Triturated with dry Et₂O precipitated 5.1 g of a solid. Recrystallization (EtOAc 125 mL-hexane 125 mL gave the title compound (4.2 g, 40%),mp 150-151° C.

Anal. Clacd. C,61.52; H, 5.94; N, 7.17; S, 8.21; F, 4.86

Found: C, 61.31; H, 5.94; N, 7.17; S, 8.14; F. 5.08

### EXAMPLE 5

Preparation of 6-[4-(1-fluoro-1-methyl-{[(methylethyl)sulfonyl]amino}ethylphenyl]-indolin-2-one (enantiomer 2).

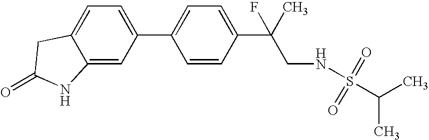

Scheme II, step A′: (−)-[2-fluoro-2-(4-iodophenyl)propyl][(methylethyl)sulfonyl]amine (0.345 g, 0.896 mmol) bis(pinacolato)diboron (0.250 g, 0.984 mmol), PdCl₂(dppf).CH₂Cl₂ (0.024 g, 0.029 mmol) and potassium acetate (0.338 g, 3.44 mmol) were heated and stirred at 80° C. in dry DMF (25.0 mL) for 3 hours under N₂. The reaction mixture was allowed to cool to ambient temperature and 6-bromo-1,3-dihydro-indol-2-one (0.209 g, 0.986 mmol), PdCl₂(dppf).CH₂Cl₂ (0.024 g, 0.029 mmol), and 2M Na₂CO₃ (2.5 mL, 5.0 mmol) were added respectively. The resulting mixture was stirred and heated at 80° C. for 6 hours. The reaction mixture was allowed to cool to ambient temperature and poured into EtOAc and extracted with H₂O. The aqueous layer was separated and extracted with EtOAc. The organic layers were combined, washed with brine and dried (MgSO₄). Filtration and concentration in vacuo gave the crude product which when chromatographed provided the final title compound, (0.025 g).

MS(ES) 391.4 (M+1)

It is understood by one of ordinary skill in the art that the racemate of examples 4 and 5, 6-[4-(1-fluoro-1-methyl-{[(methylethyl)sulfonyl]amino}ethylphenyl]-indolin-2-one, is readily prepared in a manner analogous to the procedures described therein from racemic starting material.

### EXAMPLE 6

Preparation of 6-[4-(1-fluoro-1-methyl-{[(methylethyl)sulfonyl]amino}ethylphenyl]-indolin-2-one (enantiomer 1).

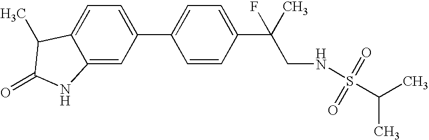

Scheme II, step A′: (+)-[2-fluoro-2-(4-iodophenyl)propyl][(methylethyl)sulfonyl]amine (0.310 g, 0.81 mmol) bis(pinacolato)diboron (0.224 g, 0.581 mmol), PdCl₂(dppf).CH₂Cl₂ (0.020 g, 0.024 mmol) and potassium acetate (0.276 g, 2.81 mmol) were heated and stirred at 80° C. in dry DMF (25.0 mL) for 3 hours under N₂. The reaction mixture was allowed to cool to ambient temperature and 6-bromo-3-methyl-1,3-dihydro-indol-2-one (0.200 g, 0.885 mmol), PdCl₂(dppf).CH₂Cl₂ (0.020 9, 0.024 mmol), and 2M Na₂CO₃ (1.5, 3.0 mmol) were added respectively. The resulting mixture was stirred and heated at 80° C. for 6 hours. The reaction mixture was allowed to cool to ambient temperature and poured into EtOAc and extracted with H₂O. The aqueous layer was separated and extracted with EtOAc. The organic layers were combined, washed with brine and dried (MgSO₄). Filtration and concentration in vacuo gave a brown residue which when chromatographed on the Chromatotron® using a 2 mm plate and eluting with a gradient EtOAc/hexane 4:6 to 6:4 system provided the final title compound, (0.0512 g, 22%). MS(ES) 405.0 (M+1).

It is understood by one of ordinary skill in the art that the racemate of example 6 and the opposite enantiomer of example 6, 6-[4-(1-fluoro-1-methyl-{[(methylethyl)sulfonyl]amino}ethylphenyl]-indolin-2-one and 6-[4-(1-fluoro-1-methyl-{[(methylethyl)sulfonyl]amino}ethylphenyl]-indolin-2-one (enantiomer 2) are readily prepared in a manner analogous to the procedure described in example 6 from racemic starting material and (−)-[2-fluoro-2-(4-iodophenyl)propyl][(methylethyl)sulfonyl]amine, respectively.

### EXAMPLE 7

Preparation of 7-[4-(1-fluoro-1-methyl-2-{[methylethyl)sulfonyl]amino}-ethyl)phenyl]indolin-2-one.

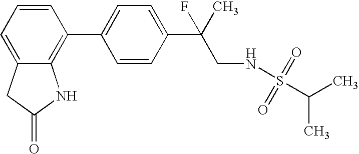

Scheme II, step A′: [2-fluoro-2-(4-iodophenyl)propyl][(methylethyl)sulfonyl]amine (0.54 g, 0.1.4 mmol), bis(pinacolato)diboron (0.40 g, 0.1.6 mmol), PdCl₂(dppf).CH₂Cl₂ (0.10 g, 0.12 mmol) and potassium acetate (0.69 g, 7.0 mmol) were heated and stirred at 80° C. in dry DMF (10.0 mL) for 3 hours under nitrogen then stirred at 23° C. for 22 h. To the reaction mixture was added 7-bromo-1,3-dihydro-indol-2-one (0.330 g, 1.6 mmol), PdCl₂(dppf).CH₂Cl₂ (CH₂Cl₂(0.10 g, 0.12. mmol), and 2M Na₂CO₃ (3.5 mL, 7.0 mmol) were added respectively. The resulting mixture was stirred and heated at 80° C. for 8 hours, then stirred at 23° C. for 21 h. The black reaction mixture was poured into H₂O (60 mL) and acidified with conc. aq. HCl(1.5 mL)forming a black sludge. The sludge was extracted with Et₂O (4×60 mL). The combined organic layers were washed with H₂O (2×60 mL), dried(Na₂SO₄) filtered, and evaporated giving 0.576 g of a brown oil. The oil was chromatographed on the Chromatotron® on a 4 mm plate eluting with EtOAc/hexane (1:1) yielding the title compound (0.157 g, 29%) as an orange red glass. MS(ES) 391.1 (M+1); 389.1 (M−1).

### EXAMPLE 8

Preparation of 5-[4-(1-fluoro-1-methyl-2-{[(methylethyl)sulfonyl]amino}-ethyl)phenyl]-3-hydrobenzimidazol-2-one

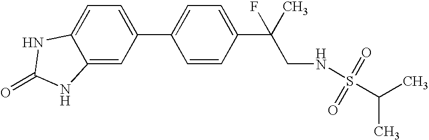

Preparation of 2-[(2-fluoro)4′-Amino-3′-nitro-biphenyl-4-yl]propyl-2-propane sulfonamide.

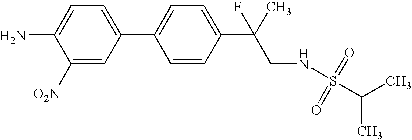

[2-fluoro-2-(4-iodophenyl)propyl][(methylethyl)sulfonyl]amine (2.00 g, 5.19 mmol), bis(pinacolato)diboron (1.45 g, 5.71 mmol), PdCl₂(dppf).CH₂Cl₂ (0.17 g, 0.21 mmol) and potassium acetate (2.40 g, 24.5 mmol) were stirred and heated under nitrogen at 80° C. in dry DMF (50 mL) for 8 h then stirred at 20° C. for 8 h. To the reaction mixture was added 4-bromo-2-nitro-aniline (1.18 g, 5.44 mmol), PdCl₂(dppf).CH₂Cl₂ (0.17 g, 0.21 mmol), and 2M Na₂CO₃(8.0 mL, 16.0 mmol). The reaction was then heated and stirred at 80° C. for 6 h. The reaction was worked up in a manner analogous to the procedure described in example 7. Flash chromatography eluting with a gradient EtOAc/hexane 2:8, 3:7 gave the intermediate title compound (1.09 g, 53%) as a red orange glass. The compound was used in the next reaction without further purification.

Preparation of 2-[(2-fluoro)3′,4′-Diamino-biphenyl-4-yl]propyl-2-propane sulfonamide.

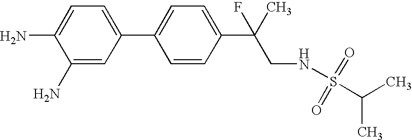

To a suspension of 10% Pd/C (0.11 g) in MeOH(10 mL) was added a solution of 2-[(2-fluoro)4′-Amino-3′-nitro-biphenyl-4-yl]propyl-2-propane sulfonamide (1.09 g, 27.6 mmol) in MeOH (60 mL) followed by a MeOH rinse (20 mL). The reaction was stirred under 1 atm hydrogen. After 92 h the reaction was complete by TLC. The reaction mixture was filtered through Celite® and the filtrate was evaporated to give the intermediate title compound (1.01 g) as a brown glass which was used without further purification.

Preparation of final title compound.

To a suspension of 2-[(2-fluoro)3′,4′-diamino-biphenyl-4-yl]propyl-2-propane sulfonamide (0.150 g, 0.412 mmol)and triphosgene(0.630 g, 2.1 mmol) in toluene (15.0 mL) was added 2M aq. HCl (15 mL) over a period of 1 minute. The resulting reaction was stirred under nitrogen for 3h. The reaction mixture was basified with solid sodium bicarbonate and filtered. The filtrate was extracted into EtOAc (50 mL). The EtOAc was dried(MgSO₄), filtered and evaporated. The residue was chromatographed several times on the Chromatotron® on a 1 mm plate eluting with EtOAc/hexane (3:2) to give the final title compound (0.031 g, 20%) as an orange glass.

### EXAMPLE 9

Preparation of 4-[4-(1-fluoro-1-methyl-2-{[methylethyl)sulfonylamino}-ethyl)phenyl]indolin-2one.

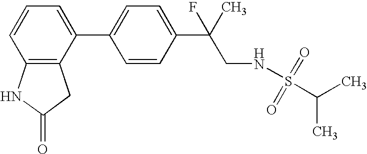

2-fluoro-2-(4-iodophenyl)propyl[(methylethyl)sulfonyl]amine (0.657 g, 1.70 mmol), bis(pinacolato)diboron (0.495 g, 1.95 mmol), PdCl₂(dppf).CH₂Cl₂ (0.072 g, 0.088 mmol) and potassium acetate (0.600g, 6.11 mmol) were heated and stirred at 80° C. in dry DMF (40.0 mL) for 3 hours under N₂. The reaction mixture was allowed to cool to ambient temperature and 4-bromo-1,3-dihydro-indol-2-one (0.440 g, 0.1.90 mmol, preparation 10), PdCl₂(dppf).CH₂Cl₂ (0.072 g, 0.088 mmol), 2M Na₂CO₃ (4.50 mL, 9.00 mmol) and additional DMF (6.00 mL) were added respectively. The resulting mixture was stirred and heated at 80° C. for 7 hours and then stirred overnight at ambient temperature. The reaction mixture was poured into H₂O (400 mL) and extracted with EtOAc (200 mL). The EtOAc layer was separated and extracted with H₂O (3×150 mL), washed with brine and filtered through Celite®. The Celite® cake was washed with additional EtOAc (2×150 mL) and the organics were combined. Evaporation in vacuo gave 0.64 g of a foam which when chromatographed on the Chromatotron® using a 4 mm plate and eluting EtOAc/hexane 1:1 system provided 0.38 g of a viscous yellow oil. Addition of Et₂O followed by evaporation gave the final title compound as a yellow solid mp 86-87° C.

Calcd. For C₂₀H₂₃FN₂O₃S.0.44 H₂O: C, 60.29; H, 6.04; N, 7.03

Found:C,60.30; H, 5.84; N, 6.81

### EXAMPLE 10

Preparation of 4-[4-(1-fluoro-1-methyl-2-{[methylethyl)sulfonylamino}-ethyl)phenyl]indolin-2one (enantiomer 1).

(+)-[2-fluoro-2-(4-iodophenyl)propyl][(methylethyl)sulfonyl]amine (0.512 g, 1.33 mmol), bis(pinacolato)diboron (0.387 g, 1.53 mmol), PdCl₂(dppf).CH₂Cl₂ (0.057 g, 0.070 mmol) and potassium acetate (0.47g, 4.79 mmol) were heated and stirred at 80° C. in dry DMF (35.0 mL) for 3 hours under N₂. The reaction mixture was allowed to cool to ambient temperature and 4-bromo-1,3-dihydro-indol-2-one (0.324 g, 0.1.53 mmol), PdCl₂(dppf).CH₂Cl₂ (0.058 g, 0.070 mmol), 2M Na₂CO₃ (3.50 mL, 7.00 mmol) and additional DMF (6.0O mL) were added respectively. T he resulting mixture was stirred and heated at 80° C. for 7 hours then stirred overnight at ambient temperature. The reaction mixture poured into H₂O (400 mL) and extracted with EtOAc (200 mL). The EtOAc layer was separated and extracted with H₂O (3×150 mL), washed with brine and filtered through Celite®. The Celite® cake was washed with additional EtOAc (2×150 mL) and the organics were combined. Evaporation in vacuo gave foam which when chromatographed on the Chromatotron® using a 4 mm plate and eluting EtOAc/hexane 1:1 system provided 0.29 g of a viscous yellow oil. Addition of Et₂O followed by evaporation on the rotary evaporator yielded the title compound (0.26 g, 50%) as an off white solid. MS(ES) 391.14 (M+1)

It is understood by one of ordinary skill in the art that enantiomer 2 of example 10 is readily prepared in a manner analogous to the procedure described therein from (+)-[2-fluoro-2-(4-iodophenyl)propyl][(methylethyl)sulfonyl]amine.

The ability of compounds of formula I to potentiate glutamate receptor-mediated response may be determined using fluorescent calcium indicator dyes (Molecular Probes, Eugene, Oregon, Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 μM Fluo3-AM dye (obtained from Molecular Probes Inc., Eugene, Oreg.) in buffer in each well. After the incubation, each well is washed once with 100 μl buffer, 200 μl of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl of 100 mM cyclothiazide to 3 ml of buffer. Control buffer solution is prepared by adding 1.5 μl DMSO to 498.5 μl of buffer.

Each test is then performed as follows. 200 μl of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5

KCl, 10 HEPES, 1 MgCl₂, 2 CaCl₂, 10 glucose, pH=7.4 with NaOH, 295 mOsm kg−1. The intracellular recording solution contains (in mM): 140 CsCl, 1 MgCl₂, 10 HEPES, (N-[2-hydroxyethyl]piperazine-N1-[2-ethanesulfonic acid]) 10 EGTA (ethylene-bis(oxyethylene-nitrilo)tetraacetic acid), pH=7.2 with CsOH, 295 mOsm kg−1. With these solutions, recording pipettes have a resistance of 2-3 MΩ. Using the whole-cell voltage clamp technique (Hamill et al.(1981)Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 μM or less, they produce a greater than 10% increase in the value of the current evoked by 1 mM glutamate.

In order to determine the potency of test compounds, the concentration of the test compound, both in the bathing solution and co-applied with glutamate, is increased in half log units until the maximum effect was seen. Data collected in this manner are fit to the Hill equation, yielding an EC₅₀ value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing.solution. In this manner, the efficacy of the test compound relative to that of cyclothiazide can be determined.

According to another aspect, the present invention provides a pharmaceutical composition, which comprises a compound of formula I or a pharmaceutically acceptable salt thereof and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 1 mg to about 500 mg, more preferably about 5 mg to about 300 mg (for example 25 mg) of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient. A more specific discussion of certain unit dosage forms are provided below followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is generally necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acidic contents of the stomach. Such formulations are created by coating a solid dosage form with a film of a polymer which is insoluble in acidic environments, and soluble in basic environments. Exemplary films are cellulose acetate phthalate, polyvinyl acetate phthalate, hydroxypropyl methylcellulose phthalate and hydroxypropyl methylcellulose acetate succinate.

Tablets are often coated with sugar as a flavor and sealant, or with film-forming protecting agents to modify the dissolution properties of the tablet. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with pores through which the compound of formula I is pumped by osmotic action.

The following formulation examples are illustrative only and are not intended to limit the scope of the invention in any way.

Formulation 1
Hard gelatin capsules are prepared using the following ingredients:

| | Quantify (mg/capsule) |
|---|---|
| Active Ingredient | 250 |
| Starch, dried | 200 |
| Magnesium Stearate | 10 |
| Total | 460 |

The above ingredients are mixed and filled into hard gelatin capsules in 460 mg quantities.

Formulation 2
Tablets each containing 60 mg of active ingredient are made as follows:

| | Quantity (mg/tablet) |
|---|---|
| Active Ingredient | 60 |
| Starch | 45 |
| Microcrystalline Cellulose | 35 |
| Polyvinylpyrrolidone | 4 |
| Sodium Carboxymethyl Starch | 4.5 |
| Magnesium Stearate | 0.5 |
| Talc | 1 |
| Total | 150 |

As used herein the term “active ingredient” refers to a compound of formula I. The active ingredient, starch, and cellulose are passed through a No. 45 mesh U.S. sieve and mixed thoroughly The solution of polyvinylpyrrolidone is mixed with the resultant powders which are then passed through a No. 14 mesh U.S. sieve. The granules so produced are dried at 50° C. and passed through a No. 18 mesh U.S. sieve. The sodium carboxymethyl starch, magnesium stearate, and talc, previously passed through a No. 60 mesh U.S. sieve, are then added to the granules which, after mixing, are compressed on a tablet machine to yield tablets each weighing 150 mg.

As used herein the term “patient” refers to a mammal, such as a mouse, guinea pig, rat, dog, horse, or human. It is understood that the preferred patient is a human.

As used herein, the terms “treating” or “to treat” each mean to alleviate symptoms, eliminate the causation either on a temporary or permanent basis, or to prevent or slow the appearance of symptoms of the named disorder. As such, the methods of this invention encompass both therapeutic and prophylactic administration.

As used herein, the term “effective amount” refers to the amount of a compound of formula I which is effective, upon single or multiple dose administration to a patient, in treating the patient suffering from the named disorder.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds can be administered by a variety of routes including oral, rectal, transdermal, subcutaneous, intravenous, intramuscular, bucal or intranasal routes. Alternatively, the compound may be administered by continuous infusion. A typical daily dose will contain from about 0.01 mg/kg to about 100 mg/kg of the active compound of this invention. Preferably, daily doses will be about 0.05 mg/kg to about 50 mg/kg, more preferably from about 0.1 mg/kg to about 25 mg/kg.

While all of the compounds of this invention are useful for potentiating glutamate receptor function in a patient, certain groups are preferred as follows:

With respect to R¹; compounds of formula I wherein R¹ is methyl, ethyl, isopropyl or N(CH₃)₂ are preferred with isopropyl being most preferred.

With respect to R², compounds of formula I wherein R² is hydrogen, methyl or ethyl are preferred, with hydrogen or methyl being most preferred.

With respect to R³, compound of formula I wherein R³ is hydrogen, methyl or ethyl are preferred, with hydrogen or methyl being most preferred.

In addition, when R² is methyl, it is most preferred that R³ is hydrogen, and when R² is hydrogen, it is most preferred that R³ is methyl.

With respect to R^{4a} and R^{4b}, compounds of formula I wherein R^{4a} and R^{4b} are each independently hydrogen, methyl, ethyl, methoxy, ethoxy, Br, Cl or F are preferred, with hydrogen, methyl, methoxy and F being most preferred.

With respect to R⁵, compounds of formula I wherein R⁵ is hydrogen or methyl are preferred.

With respect to R⁶, compounds of formula I wherein R⁶ is hydrogen or methyl are preferred.

With respect to R⁷ and R⁸, compounds of formula I wherein R⁷ and R⁸ are each independently hydrogen, methyl, or ethyl are preferred, with methyl being most preferred.

With respect to R⁹, compounds of formula I wherein R⁹ is hydrogen, methyl, or ethyl are preferred, with methyl being most preferred.

With respect to R¹⁰ and R¹¹ compounds of formula I wherein R¹⁰ is hydrogen or methyl are preferred, and R¹¹ is hydrogen or methyl are preferred. It is most preferred that R¹⁰ and R¹¹ each represent hydrogen.

With respect to Y and Z, compounds of formula I wherein Y is N when Z is O, Y is CH₂ when Z is O and Y is CH₂CH₂ when Z is O are preferred.

We claim:

1. A compound of the formula: 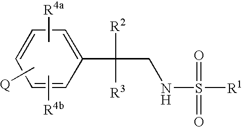
wherein
R¹ represents (1-6C)alkyl, (2-6C)alkenyl, or NR⁷R⁸;
R² and R³ each independently represent hydrogen, F, (1-4C)alkyl, or
—OR⁹;
R^{4a} and R^{4b} each independently represent hydrogen, (1-4C) alkyl, (1-4C)alkoxy, I,
Br, Cl, or F; and
Q is selected from the following: 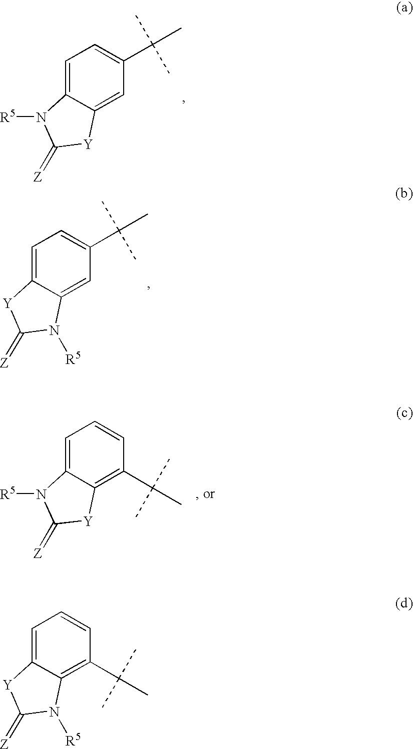
wherein
R⁵ represents hydrogen or (1-6C)alkyl;
Y represents CH₂CH₂, CR¹⁰R¹¹, NR⁶, S, or O;
Z represents O, S, or NH; and
R⁶ represents hydrogen or (1-6C)alkyl;
R⁷ and R⁸ each independently represent hydrogen or (1-4C)alkyl;
R⁹ represents hydrogen or (1-4C)alkyl; and
R¹⁰ and R¹¹ each independently represent hydrogen or (1-4C)alkyl;
or a pharmaceutically acceptable salt thereof;
with the proviso, that at least one of R² and R³ represents F, and with the further proviso that the compound of formula I is other than (±)-5-[4-(1-fluoro-1-methyl-2-{[(methylethyl)sulfonyl]amino}ethylphenyl]-indolin-2-one or (±)-6-[4-(1-fluoro-1-methyl-{[(methylethyl)sulfonyl]amino}ethylphenyl]-indolin-2-one.

2. A compound according to claim 1 wherein R¹ is (1-4C)alkyl.

3. A compound according to claim 2 wherein R¹ is isopropyl.

4. A compound according to claim 3 wherein R² is (1-4C)alkyl and R³ is hydrogen.

5. A compound according to claim 4 wherein R⁵ is hydrogen.

6. A compound according to claim 5 wherein R^{4a} and R^{4b} are each independently hydrogen, F, methyl, or methoxy.

7. A compound according to claim 6 wherein Z is O.

8. A compound according to claim 7 wherein Y is NR⁶.

9. A compound according to claim 8 wherein R⁶ is hydrogen.

10. A compound according to claim 7 wherein Y is CR¹⁰R¹¹.

11. A compound which is selected from the group consisting of:
5-[4-(1-fluoro-1-methyl-2-{[(methylethyl)sulfonyl]amino}ethylphenyl]-indolin-2-one (enantiomer 1);
5-[4-(1-fluoro-1-methyl-2-{[(methylethyl)sulfonyl]amino}ethylphenyl]-indolin-2-one (enantiomer 2);
6-[4-(1-fluoro-1-methyl-{[(methylethyl)sulfonyl]amino}ethylphenyl]-indolin-2-one (enantiomer 1);
6-[4-(1-fluoro-1-methyl-{[(methylethyl)sulfonyl]amino}ethylphenyl]-indolin-2-one (enantiomer 2);
6-[4-(1-fluoro-1-methyl-{[(methylethyl)sulfonyl]amino}ethylphenyl]-indolin-2-one;
7-[4-(1-fluoro-1-methyl-2-{[methylethyl)sulfonyl]amino}-ethyl)phenyl]indolin-2-one; and
5-[4-(1-fluoro-1-methyl-2-{[(methylethyl)sulfonyl]amino}ethyl)phenyl]-3-hydrobenzimidazol-2-one; or a pharmaceutically acceptable salt thereof.

12. A pharmaceutical composition, which comprises a compound as claimed in any one of claims 1 to 11 and a pharmaceutically acceptable diluent or carrier.

13. A method of potentiating glutamate receptor function in a patient, which comprises administering to said patient an effective amount of a compound of formula: 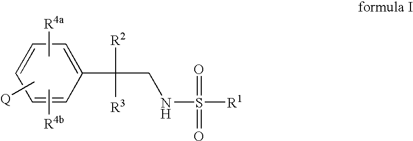
wherein
R¹ represents (1-6C)alkyl, (2-6C)alkenyl, or NR⁷R⁸;
R² and R³ each independently represent hydrogen, F, (1-4C)alkyl, or
—OR⁹;
R^{4a} and R^{4b} each independently represent hydrogen, (1-4C) alkyl, (1-4C)alkoxy, I,
Br, Cl, or F; and
Q is selected from the following: 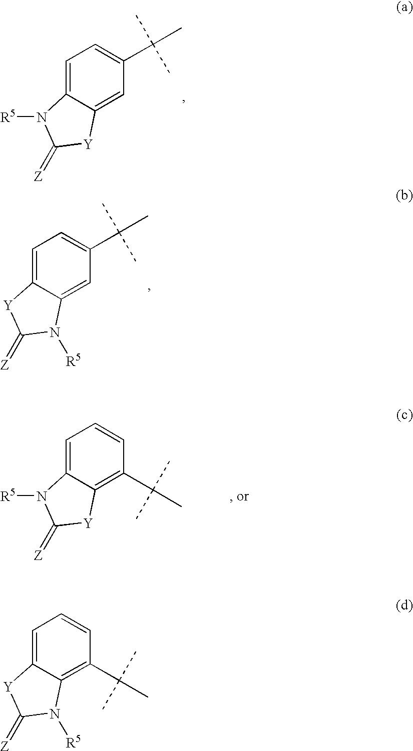
wherein
R⁵ represents hydrogen or (1-6C)alkyl;
Y represents CH₂CH₂, CR¹⁰R¹¹, NR⁶, S, or O;
Z represents O, S, or NH; and
R⁶ represents hydrogen or (1-6C)alkyl;
R⁷ and R⁸ each independently represent hydrogen or (1-4C)alkyl;
R⁹ represents hydrogen or (1-4C)alkyl; and
R¹⁰ and R¹¹ each independently represent hydrogen or (1-4C)alkyl;
or a pharmaceutically acceptable salt thereof;
with the proviso, that at least one of R² and R³ represents F, and with the further proviso that the compound of formula I is other than (±)-5-[4-(1-fluoro-1-methyl-2-{[(methylethyl)sulfonyl]amino}ethylphenyl]-indolin-2-one or (±)-6-[4-(1-fluoro-1-methyl-{[(methylethyl)sulfonyl]amino}ethylphenyl]-indolin-2-one.

14. A method of treating Alzheimer's disease in a patient, which comprises administering to said patient an effective amount of a compound of formula: 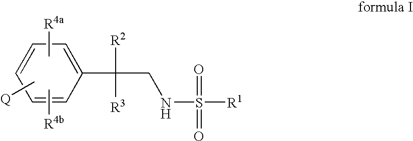
wherein
R¹ represents (1-6C)alkyl, (2-6C)alkenyl, or NR⁷R⁸;
R² and R³ each independently represent hydrogen, F, (1-4C)alkyl, or
—OR⁹;
R^{4a} and R^{4b} each independently represent hydrogen, (1-4C) alkyl, (1-4C)alkoxy, I,
Br, Cl, or F; and
Q is selected from the following: 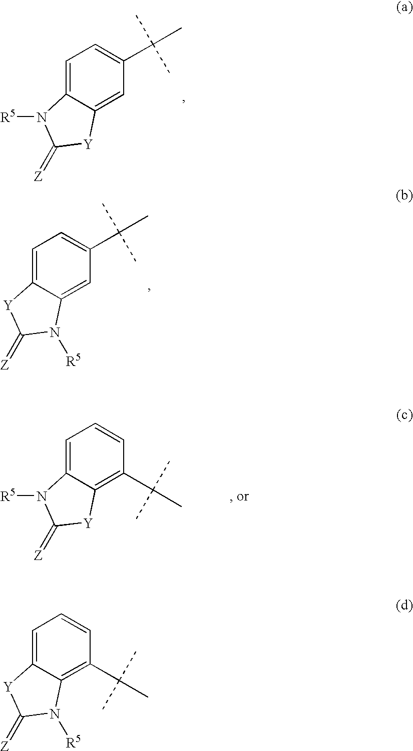
wherein
R⁵ represents hydrogen or (1-6C)alkyl;
Y represents CH₂CH₂, CR¹⁰R¹¹, NR⁶, S, or O;
Z represents O, S, or NH; and
R⁶ represents hydrogen or (1-6C)alkyl;
R⁷ and R⁸ each independently represent hydrogen or (1-4C)alkyl;
R⁹ represents hydrogen or (1-4C)alkyl; and
R¹⁰ and R¹¹ each independently represent hydrogen or (1-4C)alkyl;
or a pharmaceutically acceptable salt thereof;
with the proviso, that at least one of R² and R³ represents F, and with the further proviso that the compound of formula I is other than (±)-5-[4-(1-fluoro-1-methyl-2-{[(methylethyl)sulfonyl]amino}ethylphenyl]-indolin-2-one or (±)-6-[4-(1-fluoro-1-methyl-{[(methylethyl)sulfonyl]amino}ethylphenyl]-indolin-2-one.

15. A compound according to any of claims 1 to 11, or a pharmaceutically acceptable salt thereof, for use as a pharmaceutical.

16. The use of a compound according to any one of claims 1 to 11, or a pharmaceutically acceptable salt thereof, for the manufacture of a medicament for potentiating glutamate receptor function.

17. The use of a compound according to any one of claims 1 to 11 for the manufacture of a medicament for treating Alzheimer's disease, depression; attention deficit hyperactivity disorder; psychosis; cognitive deficits associated with psychosis; or drug-induced psychosis in a patient.
